SEQUENCE LISTING (2 sequences)

SEQ ID NO: 1 - protein, 671 aa, Primate erythroparvovirus 1
MELFRGVLQVSSNILDCANDNWWCSLLDLDTSDWEPLTHTNRLMAIYLSSVASKLDFTGGPLAGCLYFFQVECNKFEEGYHIHVVIGGPGLNPSNLTVCVEWLFNNVLYHLVTGNVKLKFLPGMTTKGKYFRDGEQFIENYLMKKIPLNVVWCVTNIDGYIDTCISATFRRGACHCQKPRLTTAINDTCNEPGESSGTGGEVVPFAGKGTKASIKFQTMVNWVCENRVFTEDKWKPVDFNQYTLLSSSHSGSFQIQSALKLAIYKATNLVPTSTFLLHTDFEQVMCIKDNKIVKLLLCQNYDPLLVGQHVLKWIDKKWGKKNTLGFYGPPSTGKTNLAMAIANNVPVYGMVNWNNENFPFNDVTGKSLVVWDEGIIKSTIVEAAKAILGGQPTRVDQKMRGSVSVPGVPVVITSNGDITFVVSGNTTTTVHAKALKERMVKLNFTVRCSPDMGLLTEADVQQWLTWCNAQSWDHYANCAINYTFDFPGINADALHPDLQTAPIVTDTSISSSGGESSEQLSESSFFNLINPGAWNTETPRSSTPIPGTSSGESFGGSSVSSEAVAASREEAFYAPLADQFRELLVGVDYVWDGVRGLPVCCVQHINNSGGGLGLCPHCINVGAWYNGWKFREFTPDLVRCSCHVGASNPFSVLTCKKCAYLSGLQSFVDYE

SEQ ID NO: 2 - protein, 671 aa, Primate erythroparvovirus 1
  Variant 385: Xaa = Any amino acid
MELFRGVLHISSNILDCANDNWWCSMLDLDTSDWEPLTHSNRLIAIYLSSVASKLDFTGGPLAGCLYFFQVECNKFEEGYHIHVVIGGPGLNARNLTVRVEGLFNNVLYHLVTETVKLKFLPGMTTKGKYFRDGEQFIENYLMKKIPLNVVWCVTNIDGYIDTCISASFRRGACHAKRPRITANTDNVTSETGESSCGGGDVVPFAGKGTKAGLKFQTMVNWLCENRVFTEDKWKLVDFNQYTLLSSSHSGSFQIQSALKLAIYKATSLVPTSTFLLHSDFEQVTCIKDNKIVKLLLCQNYDPLLVGQHVLKWIDKKCGKKNTLWFYGPPSTGKTNLAMAIAKTVPVYGMVNWNNENFPFNDVAGKSLVVWDEGIIKSTIVEAAXAILGGQPTRVDQKMRGSVAVPGVPVVITSNGDITFVVSGNTTTTVHAKALKERMVKLNFTVRCSPDMGLLTEADVQQWLTWCNAQSWNHYENWAINYTFDFPGINADALHPDLQTTPIVPDTSISSSGGESSEELSESSFFNLITPGAWNSETPRSSTPVPGTSSGESSVGSPVSSEVVAASWEEAFYTPLADQFRELLVGVDFVWDGVRGLPVCCVEHINNSGGGLGLCPHCINVGAWYNGWKFREFTPDLVRCSCHVGASNPFSVLTCKKCAYLSGLQSFVDYE

(19) United States
(12) Patent Application Publication
(10) Pub. No.: US 2014/0248285 A1
(43) Pub. Date: Sep. 4, 2014

(54) METHOD FOR PROTECTING PARVOVIRUS ANTIGEN

(71) Applicants: Michael Broeker, Marburg (DE); Susanne Modrow, Regensburg (DE); Simon Bredl, Schlieren (CH)

(72) Inventors: Michael Broeker, Marburg (DE); Susanne Modrow, Regensburg (DE); Simon Bredl, Schlieren (CH)

(73) Assignee: Novartis AG, Basel (CH)

(21) Appl. No.: 14/002,701

(22) PCT Filed: Mar. 2, 2012
(86) PCT No.: PCT/IB2012/051007

§ 371 Date: Apr. 28, 2014

Related U.S. Application Data
(60) Provisional application No. 61/464,469, filed on Mar. 3, 2011.

(51) Int. Cl. G01N 33/569

(52) U.S. Cl. CPC G01N 33/56983
USPC 424/159.1; 435/5; 514/13.7; 514/15.2

(57) ABSTRACT

An improved method for the detection of parvovirus B19 in a sample is provided, the improvement consisting of detecting a parvovirus B19 non-structural protein in said sample.

### TECHNICAL FIELD

This invention is in the field of detection and research for human parvovirus B19.

### BACKGROUND ART

Parvovirus B19 (B19V) is a small non-enveloped virus with a single-stranded DNA genome of approximately 5,600 nucleotides (see review articles 1-5). It has at least 3 known genotypes. The virus particles consist of two structural proteins (VP1 and VP2). In addition to the two structural proteins, the genome encodes two non-structural proteins, NS1 and NS2. NS1 (77 kDa) is a multifunctional protein which is produced in infected cells during viral replication and is not part of the infectious virus particle (6). Synthesis of all nine viral genome transcripts is controlled by a single promoter which is located at map unit 6 (p6) and is activated by the viral NS1 protein (7-9). Only the NS1 transcript is non-spliced; the eight others, including the two capsid proteins (VP1 and VP2) are generated by a series of different splicing events (6, 10, 11). In addition to transactivator, helicase and endonuclease activities, which are essential for viral genome replication, it has properties which induce apoptosis (12-15).

Parvovirus B19 infects humans, and the incubation time of the infection is on average one to two weeks. In this phase the patient is already viraemic and can transmit the virus. The most common appearance of the disease is Erythema infectiosum, also known as “fifth disease” (4). Erythema infectiosum occurs mainly in infants and is characterized by symptoms similar to flu with light fever. These are accompanied by an exanthema which occurs first on the cheeks and then spreads during the course of the disease on the inner sides of arms and legs and lasts for one to two days. Infection can also cause arthralgies and severe inflammation of the joints which last for several weeks, or even years after infection and often resemble rheumatoid arthritis. In some patients other autoimmune diseases like vasculitis, Hashimoto thyroiditis and autoimmune anemias, neutropenias and thrombopenias can develop after the acute infection (see review articles 5, 16, 17).

When parvovirus B19 infects pregnant women, it can be diaplacentally transmitted to the fetus and cause severe, sometimes deadly diseases. During the first trimester an acute parvovirus B19 infection can cause spontaneous abortion; until the 20th week of pregnancy it can lead to the establishment of a Hydrops fetalis. In one third of infections the virus is diaplacentally transmitted to the embryo with a delay of several weeks to acute infection of the pregnant woman, mainly during the second but also at the start of the third trimester. It infects mainly the pronormoblasts of the embryo's liver. Severe anemias, circulatory disorders and Hydrops fetalis are the consequences (see reviews 1, 18, 19).

The detection of the B19 virus in biological material (e.g. blood, serum or tissue) is required for the diagnosis of acute and persisting parvovirus B19 infections. This is currently achieved by quantitative or qualitative detection of the virus genome with DNA detection methods like PCR or Southern blot (20). However, detection of viral DNA allows no conclusion with respect to the infectious potential of a sample as the number of genomes present does not correspond to the number of infectious units because of the potential presence of free DNA and/or virus particles containing defective viral genomes in the sample material. Reference 21 detected parvovirus B19 DNA in blood plasma products but the authors note that they were not able to determine the infectivity of the plasma products because various methods for virus inactivation are applied during the manufacturing process of plasma products and the detection of viral DNA cannot be equated with infectious particles.

There is thus a need for improved methods for detecting parvovirus B19. Methods for detecting other parvoviruses are known, but differences within the parvoviridae family mean that these methods are of limited relevance. Parvovirus B19 infects humans exclusively and no animal infection model exists. Other members of the parvoviridae family infect mainly the enterocytes of other mammals (e.g. porcine parvovirus and canine parvovirus) but these viruses are not of the same genus as B19, which is in the erythrovirus genus.

Current methods for the detection of replication-competent parvovirus B19V involve propagation in cell culture followed by detection of viral mRNA species, of intermediate products such as genome dimers which occur during the replication of the virus genome, of or viral structural proteins in the infected cells or the culture supernatant. These methods have proven to be ineffective for assessing the number of infectious units in a sample because, for instance, the viral structural proteins of the inoculum used for the infection overlay the detection of the newly formed structural proteins. The same is true for the detection of the viral transcripts. Also, it is not possible to distinguish between non-spliced mRNA and viral genomes after reverse transcription (22-24). Therefore, current methods for detecting parvovirus B19 are not suitable for any assay or analysis that requires the specific detection of replication-competent parvovirus B19.

Therefore, there is a requirement for new and improved analytical methods for detecting infectious, replication-competent parvovirus B19.

### DISCLOSURE OF THE INVENTION

The invention permits detection of replication-competent parvovirus B19 by detecting non-structural viral proteins. These proteins arise only from replication-competent viruses and so the results of the methods are not obscured by defective virus particles. Moreover, the method is not confounded by any free DNA in the sample. As demonstrated in the Examples, the method of the invention is able to distinguish between samples that comprise the same amount of parvovirus B19 DNA but different amounts of infectious particles. In addition, the method of the invention does not require the isolation of viral nucleic acids. The isolation of viral mRNA transcripts, as an indicator of active virus replication, in particular is prone to complex and time consuming experimental procedures. Both alternative RNA-splicing and RNA-degradation may exert major influences on the quantification of viral mRNAs, thereby resulting in miscalculations of infectious units. Furthermore, as shown herein, detection of non-structural proteins does not interfere with or inhibit infection of cells with parvovirus B19, in contrast to antibodies against the two structural proteins. Thus methods of the invention can permit detection of parvovirus B19 without interfering with the process of infection, which is useful for the unequivocal detection of replication-competent parvovirus B19 and for accurate analysis of modulators of parvovirus B19 infectivity. Thus the methods allow improved and accurate detection of replication-competent parvovirus B19.

In general terms, therefore, the invention provides, in a method for the detection of parvovirus B19 in a sample, the improvement consisting of detecting a parvovirus B19 non-structural protein.

The invention also provides a method for the detection of parvovirus B19 in a sample, comprising steps of: (i) contacting the sample with cells which can be infected by parvovirus B19; (ii) incubating the cells; and (iii) determining the presence of parvovirus B19 non-structural proteins.

The invention also provides a method for the diagnosis and/or confirmation of parvovirus B19 infection in a subject, comprising a step of detecting parvovirus B19 non-structural proteins in a sample from the subject. This method is preferably an in vitro method.

The invention also provides kits for detecting parvovirus B19, comprising a reagent (e.g. an antibody) for detecting a non-structural protein (e.g. NS1). In certain embodiments, the kits can include a source of cells which support replication of parvovirus B19.

The invention also provides an antibody that specifically detects a parvovirus B19 non-structural protein (e.g. an anti-NS1 antibody) for use in detecting parvovirus B19 and/or for use in diagnosis of parvovirus B19 infection. Suitable antibodies are disclosed in reference 25 e.g. the hMab1424 antibody whose amino acid sequence is available as GenInfo identifier GI:3747019 (light chain variable region) and GI:3747018 (heavy chain variable region).

A method for the detection of parvovirus B19 according to the invention is advantageously a method for the detection of replication-competent parvovirus B19. In some embodiments a method for the detection of parvovirus B19 according to the invention is for detecting infectious particles. In some embodiments a method for the detection of parvovirus B19 according to the invention is for detecting virus particles that have not been inactivated, or that have not been neutralised.

Recombinant non-structural parvovirus proteins have been used to detect anti-NS antibodies in animal sera. Such methods can be used to distinguish animals that have been infected with a virus from animals that have been vaccinated with inactivated virus particles. Only animals that have been infected with the virus will have antibodies against non-structural proteins because vaccines generally comprise structural envelope proteins only. As there is no vaccine for parvovirus B19, however, such methods have not been considered for use in relation to parvovirus B19. Furthermore, these methods use NS proteins as reagents for detecting anti-NS antibodies, whereas methods of the present invention use detection of NS proteins to assess the presence or absence of virus.

In addition, methods and reagents relating to parvoviruses that infect animals are of limited relevance to methods for the detection of parvovirus B19. Parvovirus B19 differs significantly from other parvoviruses in its target cells, host, cellular receptor, transcription profile, capsid structure, stability, the externalisation of its DNA, its VP2 cleavage, the exposure of the N-terminal of VP1 and in many other features of its activity and function (26-30).

The invention can be used to detect any of genotype 1, 2 and/or 3 of B19.

### The Non-Structural Protein

The invention can use non-structural protein NS1 and/or non-structural protein NS2. In preferred embodiments the method is based on NS1.

Various amino acid sequences are known for NS1 from B19 parvoviruses. The full-length protein is typically a 671-mer (e.g. GI:49616867 and GI:86211074) but shorter fragments have been reported in various types of sample e.g. a 95-mer sequence from skeletal muscle (GI:12060988).

The sequence is not 100% conserved between different isolates e.g. the 671-mer NS1 sequences from the Vn147 isolate (GI:86211068; SEQ ID NO: 1) and the Br543 isolate (GI:49616867; SEQ ID NO: 2) have 615/671 identical residues (92% identity):

| Score = 1200 bits (3104), Expect = 0.0, Method: Compositional matrix adjust. | | | |
|---|---|---|---|
| Identities = 615/671 (92%), Positives = 637/671 (95%), Gaps = 0/671 (0%) | | | |
| SEQID2 | 1 | MELFRGVLHISSNILDCANDNWWCSMLDLDTSDWEPLTHSNRLIAIYLSSVASKLDFTGG | 60 |
| | | MELFRGVL +SSNILDCANDNWWCS+LDLDTSDWEPLTH+NRL+AIYLSSVASKLDFTGG | |
| SEQID1 | 1 | MELFRGVLQVSSNILDCANDNWWCSLLDLDTSDWEPLTHTNRLMAIYLSSVASKLDFTGG | 60 |
| SEQID2 | 61 | PLAGCLYFFQVECNKFEEGYHIHVVIGGPGLNARNLTVRVEGLFNNVLYHLVTETVKLKF | 120 |
| | | PLAGCLYFFQVECNKFEEGYHIHVVIGGPGLN RNLTV VEGLFNNVLYHLVT VKLKF | |
| SEQID1 | 61 | PLAGCLYFFQVECNKFEEGYHIHVVIGGPGLNPRNLTVCVEGLFNNVLYHLVTGNVKLKF | 120 |
| SEQID2 | 121 | LPGMTTKGKYFRDGEQFIENYLMKKIPLNVVWCVTNIDGYIDTCISASFRRGACHAKRPR | 180 |
| | | LPGMTTKGKYFRDGEQFIENYLMKKIPLNVVWCVTNIDGYIDTCISA+FRRGACH ++PR | |
| SEQID1 | 121 | LPGMTTKGKYFRDGEQFIENYLMKKIPLNVVWCVTNIDGYIDTCISATFRRGACHCQKPR | 180 |
| SEQID2 | 181 | ITANTDNVTSETGESSCGGGDVVPFAGKGTKAGLKFQTMVNWLCENRVFTEDKWKLVDFN | 240 |
| | | +T ++ E GESS GG+VVPFAGKGTKA +KFQTMVNWLCENRVFTEDKWK VDFN | |
| SEQID1 | 181 | LTTAINDTCIEAGESSGTGGEVVPFAGKGTKASIKFQTMVNWLCENRVFTEDKWKPVDFN | 240 |
| SEQID2 | 241 | QYTLLSSSHSGSFQIQSALKLAIYKATSLVPTSTFLLHSDFEQVTCIKDNKIVKLLLCQN | 300 |
| | | QYTLLSSSHSGSFQIQSALKLAIYKAT+LVPTSTFLLH+DFEQV CIKDNKIVKLLLCQN | |
| SEQID1 | 241 | QYTLLSSSHSGSFQIQSALKLAIYKATNLVPTSTFLLHTDFEQVMCIKDNKIVKLLLCQN | 300 |
| SEQID2 | 301 | YDPLLVGQHVLKWIDKKCGKKNTLWFYGPPSTGKTNLAMAIAKTVPVYGMVNWNNENFPF | 360 |
| | | YDPLLVGQHVLKWIDKKCGKKNTLWFYGPPSTGKTNLAMAIAK+VPVYGMVN +NENFPF | |
| SEQID1 | 301 | YDPLLVGQHVLKWIDKKCGKKNTLWFYGPPSTGKTNLAMAIAKSVPVYGMVNGHNENFPF | 360 |
| SEQID2 | 361 | NDVAGKSLVVWDEGIIKSTIVEAAXAILGGQPTRVDQKMRGSVAVPGVPVVITSNGDITF | 420 |
| | | NDV GKSLVVWDEGIIK TIVEAA AILGGQPTRVDQKMRGSV VPGVPVVITSNGDITF | |
| SEQID1 | 361 | NDVPGKSLVVWDEGIIKCTIVEAAKAILGGQPTRVDQKMRGSVPVPGVPVVITSNGDITF | 420 |
| SEQID2 | 421 | VVSGNTTTTVHAKALKERMVKLNFTVRCSPDMGLLTEADVQQWLTWCNAQSWNHYENWAI | 480 |
| | | VVSGNTTTTVHAKALKERMVKLNFTVRCSPDMGLLTEADVQQWLTWCNAQSW+HY N AI | |
| SEQID1 | 421 | VVSGNTTTTVHAKALKERMVKLNFTVRCSPDMGLLTEADVQQWLTWCNAQSWDHYANCAI | 480 |
| SEQID2 | 481 | NYTFDFPGINADALHPDLQTTPIVPDTSISSSGGESSEELSESSFFNLITPGAWNSETPR | 540 |
| | | NYTFDFPGINADALHPDLQT PIV DTSISSSGGESSE+LSESSFFNLI PGAWN+ETPR | |
| SEQID1 | 481 | NYTFDFPGINADALHPDLQTAPIVTDTSISSSGGESSEQLSESSFFNLINPGAWNTETPR | 540 |
| SEQID2 | 541 | SSTPVPGTSSGESSVGSPVSSEVVAASWEEAFYTPLADQFRELLVGVDFVWDGVRGLPVC | 600 |
| | | SSTP+PGTSSGES GS VSSE VAAS EEAFY PLADQFRELLVGVD+VWDGVRGLPVC | |
| SEQID1 | 541 | SSTPVPGTSSGESFGGSSVSSEAVAASREEAFYAPLADQFRELLVGVDYVWDGVRGLPVC | 600 |
| SEQID2 | 601 | CVEHINNSGGGLGLCPHCINVGAWYNGWKFREFTPDLVRCSCHVGASNPFSVLTCKKCAY | 660 |
| | | CV+HINNSGGGLGLCPHCINVGAWYNGWKFREFTPDLVRCSCHVGASNPFSVLTCKKCAY | |
| SEQID1 | 601 | CVQHINNSGGGLGLCPHCINVGAWYNGWKFREFTPDLVRCSCHVGASNPFSVLTCKKCAY | 660 |
| SEQID2 | 661 | LSGLQSFVDYE | 671 |
| | | LSGLQSFVDYE | |
| SEQID1 | 661 | LSGLQSFVDYE | 671 |

The invention can look at any part of NS1 but preferably looks at a sequence which is well conserved between different isolates and/or genotypes e.g. as shown in the above alignment.

Methods of the invention are effective with any technique for detection of proteins, including but not limited to immunoblotting (e.g. western blotting), immunoprecipitation, immunoelectrophoresis, mass-spectrometry, immunodiffusion (e.g. SRID), immunochemical methods, binder-ligand assays (e.g. ELISA), immunohistochemical techniques, agglutination assays, etc.

Immunoassay methods are preferred, in which non-structural protein is detected by using one or more antibodies. Antibodies useful in these methods may be specific for any part of a parvovirus B19 non-structural protein but, as mentioned above, are ideally specific for a sequence which is well conserved between isolates and/or genotypes. The differences between B19 genotypes 1, 2 and 3 are mostly located in the region encoding the carboxyterminal part of the NS1 protein and so in certain embodiments the methods of the invention use antibodies specific for other regions of the protein. Other methods may use antibodies specific for the C-terminal portion of the NS1 protein e.g. in order to distinguish different genotypes from each other. In some embodiments the antibody is monoclonal antibody 1424 (25). Various immunoassay formats are available to the skilled person and these often involve the use of a labelled antibody e.g. with an enzymatic, fluorescent, chemiluminescent, radioactive, or dye label. Assays which amplify signals from immune complexes are also known e.g. those which utilize biotin and avidin, and enzyme-labelled and mediated immunoassays, such as ELISA.

The “antibody” used in these methods can take various forms. Thus the antibody may be a polyclonal or monoclonal preparation. For specificity and reproducibility reasons it is preferred to use a monoclonal antibody. The antibody may be native antibodies, as naturally found in mammals, or artificial. Thus the antibody may be, for example, a fragment of a native antibody which retains antigen binding activity (e.g. a Fab fragment, a Fab′ fragment, a F(ab′)₂ fragment, a Fv fragment), a “single-chain Fv” comprising a V_{H} and V_{L} domain as a single polypeptide chain, a “diabody”, a “triabody”, a single variable domain or VHH antibody, a “domain antibody” (dAb), a chimeric antibody having constant domains from one organism but variable domains from a different organism, a CDR-grafted antibody, etc. The antibody may include a single antigen-binding site (e.g. as in a Fab fragment or a scFv) or multiple antigen-binding sites (e.g. as in a F(ab′)₂ fragment or a diabody or a native antibody). Where an antibody has more than one antigen-binding site it is preferably a mono-specific antibody i.e. all antigen-binding sites recognize the same antigen.

An antibody may include a non-protein substance e.g. via covalent conjugation. For example, an antibody may include a detectable label.

The term “monoclonal” as originally used in relation to antibodies referred to antibodies produced by a single clonal line of immune cells, as opposed to “polyclonal” antibodies that, while all recognizing the same target protein, were produced by different B cells and would be directed to different epitopes on that protein. As used herein, the word “monoclonal” does not imply any particular cellular origin, but refers to any population of antibodies that all have the same amino acid sequence and recognize the same epitope(s) in the same target protein(s). Thus a monoclonal antibody may be produced using any suitable protein synthesis system, including immune cells, non-immune cells, acellular systems, etc. This usage is usual in the field e.g. the product datasheets for the CDR-grafted humanised antibody Synagis™ expressed in a murine myeloma NS0 cell line, the humanised antibody Herceptin™ expressed in a CHO cell line, and the phage-displayed antibody Humira™ expressed in a CHO cell line all refer the products as monoclonal antibodies. The term “monoclonal antibody” thus is not limited regarding the species or source of the antibody, nor by the manner in which it is made.

An antibody used with the invention is ideally one which can bind to a parvovirus NS1 sequence consisting of SEQ ID NO: 1 and/or to a parvovirus NS1 sequence consisting of SEQ ID NO: 2. These antibodies can bind to many different NS1 sequences for a variety of strains and isolates.

The NS1 protein to be detected will usually (i) have at least w % sequence identity to SEQ ID NO: 1 and/or (ii) comprise of a fragment of at least x contiguous amino acids from SEQ ID NO: 1. The value of w is at least 85 (e.g. 86, 87, 88, 89, 90, 91, 92, 93, 94, 95, 96, 97, 98, 99 or more). The value of x is either at least 7 (e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 250, 300) and the fragment will usually include an epitope from SEQ ID NO: 1. The NS1 protein will usually be able to bind to an antibody which can bind to a parvovirus NS1 sequence consisting of SEQ ID NO: 1.

The non-structural protein may be determined in the presence or absence of cells, and may be determined in intracellular or extracellular form. For instance, in some embodiments a method can comprise determining the number of cells in a culture which are positive for expression of the non-structural protein. This protein expression shows that the cell was infected by a replication-competent B19V virus. In other embodiments the amount of non-structural protein produced by a population of cells is determined. These measurements can be used to determine the presence and/or quantity of replication-competent B19 parvoviruses in the sample.

Cells which express the non-structural protein can be determined using flow cytometry e.g. by using fluorescence-activated cell sorting (FACS) techniques. Such methods allow rapid determination of the number of cells positive for the non-structural protein and, therefore, rapid evaluation of the replication-competent virus particles in the biological sample being tested.

This application refers to steps of detecting or determining the presence of non-structural proteins. It will be appreciated that this refers to a step which is suitable for detecting non-structural proteins which might be present. If no such proteins are present in the sample then the detection step will give a negative result, but the method has still involved detecting the non-structural proteins. Thus the step encompasses detection of both the presence and absence of the non-structural proteins.

In some embodiments the methods of the invention are for providing a qualitative analysis of parvovirus B19 in a sample (e.g. presence/absence). In other embodiments the methods of the invention are for providing a semi-quantitative analysis of parvovirus B19 infection. In other embodiments the methods of the invention are for providing a quantitative analysis of parvovirus B19 infection. In other embodiments the methods of the invention are for measuring the infectivity of a sample of parvovirus B19. In other embodiments the methods of the invention are for measuring the permissivity of a population of cells to parvovirus B19 infection.

### The Sample

The sample tested with the methods of the invention can be any sample that contains (or is suspected to contain, or which might contain) parvovirus B19.

In some embodiments the sample is a biological sample such as blood, serum, plasma, sputum, saliva, amniotic fluid, synovial fluid, cerebrospinal fluid, follicular fluid, ascites fluid or any tissue. In preferred embodiments, the sample is a blood plasma product such as a coagulation factor concentrate, serum albumin, or an immunoglobulin preparation.

In some embodiments the sample tested is a non-biological sample that might be contaminated with parvovirus B19.

In certain embodiments the sample tested is or is from a pharmaceutical product. For instance, the product may be a parvovirus B19 vaccine composition, a vaccine composition which includes a parvovirus B19 component, or a blood plasma product (e.g. see below).

The sample may be a heat-inactivated sample, or a sample from a heat-inactivated product.

The methods of the invention are useful for detecting replication-competent parvovirus both in samples obtained from patients suspected of being infected with parvovirus B19, and in samples from products that are to be administered to a human and which thus should be certified to be free of parvovirus B19.

Methods of the invention do not have to be performed on a complete sample. Thus a sample can be obtained, and the method can be performed on a portion of the sample e.g. on portions of a biopsy, or on aliquots of a cell culture sample.

A patient sample will generally be from a human patient. The human may have a symptom of parvovirus B19 infection e.g. they may be anemic (for example sickle cell disease, thalassaemia, Fanconi anemia), including aplastic anemia; they may have thrombocytopenias and/or neutropenias; they may have hepatitis and/or myocarditis; they may have encephalitis.

Quantitative measurement of NS1 in a sample can be used to determine the number of infectious units present in the original material. For instance, serial dilutions of a sample can be used to assist in determining the number of infectious units present in the sample. The B19V structural proteins or the B19V DNA present in a test sample may be quantified, for example by qPCR, to assist in quantifying the parvovirus B19 present in the original sample and to assist in preparing diluted samples for an assay. The assay can be calibrated using any suitable positive control e.g. using a composition known to include only infectious viruses with no free DNA, whose titre has been assessed by qPCR.

### Cells Which Can Be Infected by B19

Methods of the invention can involve contacting a sample with cells which can be infected by parvovirus B19. If the sample contains replication-competent virus then it can infect the cells and cause them to express the non-structural proteins. Thus the cells are used under conditions suitable for their infection of the cells by parvovirus B19. Such conditions are known to the skilled person and suitable conditions are provided in the examples.

The methods of the invention are compatible with any cell that can be infected by parvovirus B19, including any of the cells described below. The cellular receptor that mediates the entry of parvovirus B19 into its target cells is globoside P (blood group antigen P) and so cells used with methods of the invention will typically express globoside P on their surface. Suitable cells include, but are not limited to, human erythroid progenitor cells (EPCs), colony-forming unit erythroids (CFU-E), burst forming unit erythroids (BFU-E), erythroblasts (particularly those in bone marrow), erythroleukemia cell lines such as JK-1 (31, 32) and KU812Ep6 (33), and megakaryoblastoid cell lines, such as MB02 (34), UT7/Epo (35) and UT7/Epo-S1, a sub-clone of UT7/Epo (36). In preferred embodiments, the cells are CD36⁺ EPCs.

A comparative study of a number of different cells regarding the permissitivity to B19V infection demonstrated that UT7/Epo-S1 cells are most sensitive to B19V replication and expression (37).

Erythroid progenitor cells generated ex vivo, which can be obtained from bone marrow cells, are a suitable, permissive system for B19V replication (38-40). These progenitor cells are also present in peripheral blood (41), in umbilical cord blood (42) and in fetal liver (43, 44).

Wong et al. (45) showed that large numbers of permissive EPCs can be generated from hematopoietic stem cells (HSCs) (46, 47) by using a cell culture system that allow the differentiation and expansion of CD34⁺ HSCs into CD36⁺ EPCs. Then Filippone et al. (48) continued the further development of this system and showed that CD36⁺ EPCs can be generated from peripheral blood mononuclear cells (PBMCs) without an in vitro preselection of CD34⁺ HSCs. It was also shown that these CD36⁺ EPCs express the B19V cellular receptor globoside P (GloP) on their cell surfaces and are highly permissive to B19V infection.

Reference 49 demonstrates that endothelial progenitor cells positive for KDR and/or CD133 and/or CD34 are permissive of parvovirus B19 infection.

The methods of the invention can be used to identify other cells and cell lines that are permissive of parvovirus B19 infection and to determine whether or not a particular cell or cell line is permissive of parvovirus B19 infection. In such embodiments, the detection of non-structural proteins indicates that the cell or cell line used to contact the sample comprising parvovirus B19 is permissive to parvovirus B19 infection.

### Testing of Viral Inactivation and Antiviral Agents

In certain embodiments the method of the invention is used to evaluate the effectiveness of a method for inactivation or destruction of parvovirus B19. In such embodiments the sample can be an artificially prepared parvovirus B19 sample that may or may not have been exposed to a certain treatment. Due to its molecular properties, parvovirus B19 is very stable and resistant to inactivation methods like pasteurization, detergent and heat treatment. By applying the method of the invention different methods of potential inactivation can be quickly and unequivocally evaluated. Such a use is demonstrated in Example 5 where the ability of heating to inactivate parvovirus B19 was analysed.

The invention also provides a method for verifying the inactivation of parvovirus B19 in a composition, comprising performing the detection method of the invention on the composition or on a sample thereof. If parvovirus is detected then this result indicates that the inactivation has failed.

Similarly, the methods of the invention can be used to determine the effectiveness of parvovirus B19 neutralizing antibodies or the presence of such antibodies in patients with persisting infection. In certain embodiments, the sample to be analysed is pre-treated with a preparation of B19-specific antibodies or serum or plasma samples which may contain parvovirus B19-specific antibodies. Alternatively, the sample comprising parvovirus, the sample comprising antibodies, and the population of cells can be co-incubated. Using the methods of the invention, the presence and effectiveness of B19 neutralizing immunoglobulins in the serum or plasma sample or the preparation used for pre-treatment can be determined by assessing how the infectivity of the parvovirus B19 is affected by the pre-treatment. This is a prerequisite for the rational application of immunoglobulin preparations for therapy of persisting parvovirus B19 infections. Such a use is demonstrated in Example 3 where the neutralising ability of antibodies specific for VP1 and VP2 was demonstrated and in Example 4 where the presence of neutralising antibodies in different sera was compared.

The methods of the invention can also be used to detect and characterise parvovirus B19 neutralizing antibodies present in samples from convalescent patients or from vaccinated subjects. Therefore the methods of the invention will be useful in the development of vaccines against parvovirus B19 infection. The genes of the viral structural proteins or sections thereof can be expressed in different prokaryotic and eukaryotic systems. In this way it is possible to produce virus-like particles or the viral structural proteins VP1 and VP2 or parts thereof, to purify them and to use them for inoculation in test animals or volunteers. Through application of the method of the invention, it can be determined whether and to what extent the different viral proteins or sections of proteins are able to induce the formation of neutralizing immunoglobulins.

### Methods of Testing Pharmaceutical Products

In certain embodiments, the invention provides a method of testing a pharmaceutical product comprising contacting the product (or a sample thereof) with a population of cells and detecting a parvovirus B19 non-structural protein.

The method is useful for certifying that a product is free from parvovirus B19 or, more specifically, from replication-competent parvovirus B19.

The invention additionally provides a pharmaceutical product such as a parvovirus B19 vaccine composition that has been tested using the methods of the invention and that is free from parvovirus B19.

The product may be a heat-inactivated product.

The product may contain human serum albumin.

### Methods of Manufacturing Blood Products

Due to the resistance of parvovirus B19 to inactivation procedures [21], blood products are at risk of being contaminated by parvovirus B19. The invention provides improved methods for the manufacture of blood products comprising contacting the product or a sample thereof with suitable cells and detecting a non-structural protein. Such methods can be used to accept blood samples that are free from parvovirus B19 for inclusion in a blood product. Such methods can be used to reject samples that are detected to be positive for parvovirus B19. Therefore, the methods of manufacture can incorporate a screening step comprising detecting a non-structural protein.

The invention additionally provides blood products that are produced by the manufacturing methods of the invention or that are certified to be free of parvovirus B19 using methods of the invention.

Blood products which can be tested using the invention include, but are not limited to: whole blood; plasma (e.g. apheresis plasma or recovered plasma); serum; platelets; blood plasma products; coagulation factor concentrate; coagulation factors such as factors VII, VIII, IX, or factor VIII/vWF; activated prothrombin complex concentrate (APCC) serum albumin, including human serum albumin; or immunoglobulin preparations. The product may be a heat-inactivated product.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 50-56, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 57. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 58.

The word “substantially” does not exclude “completely” e.g. a composition which is “substantially free” from Y may be completely free from Y. Where necessary, the word “substantially” may be omitted from the definition of the invention.

### BRIEF DESCRIPTION OF THE FIGURES

FIG. 1. In vitro differentiation of human peripheral blood cells to CD36+ erythroid progenitor cells. FACS analysis of cells at day 0 and day 10 of cultivation in expansion medium.

FIG. 2. FACS analysis of CD36+ erythroid precursor cells generated in vitro. Analysis of B19V NS1 expression in CD36+/Globoside P+ (GloP) cells 24 hours post infection. Upper panel: Erythroid progenitor CD36+ cells not infected with parvovirus B19. Lower panel: Erythroid progenitor CD36+ cells infected with parvovirus B19 (MOI (multiplicity of infection) of 1000/cell)

FIG. 3. Analysis of the influence of the incubation time after infection (24 h, 48 h and 72 h) and of the MOT/cell (0.1 to 1000 MOI/cell) on the percentage of NS1-positive cells in the respective cultures.

FIG. 4. Analysis of the neutralisation capacity of the monoclonal antibodies hmab1424 (NS1-specific), hmab1418 (VP1-specific) and hmab860-55 (VP2-specific). Erythroid progenitor CD36+ cells were generated in vitro. After 10 days of differentiation, CD36+ cells infected with parvovirus B19 (MOT 1000/cell). The virus inoculum was incubated with various concentrations (0-10 μg/ml) of the respective purified monoclonal antibodies. Cells were analyzed for B19V NS1 expression 24 hours post infection.

FIG. 5. Calculation of the neutralisation IC50 values of the monoclonal antibodies 860-55, 1418 and 1424, analysed as described for FIG. 4.

FIG. 6. Determination of the parvovirus B19 neutralizing capacity of antibodies present in sera from seropositive (+) donors and a seronegative (−) donor. Cells were infected with a MOT of 1000 B19V and were co-incubated with different dilutions of the sera from seropositive (+) donors and a single dilution of the sera from a seronegative donor (−). For each dilution the bars represent sera 1-5 from left to right. B19V NS1 expression was analyzed 24 h post infection. The controls were cells incubated with no sera (positive control), and cells that were not infected (negative control).

FIG. 7. Analysis of the impact of heat treatment of viremic plasma upon B19V infectivity. The plasma was incubated for 5 min at the indicated temperature. The cells were infected with a MOI of 1000 and B19V NS1 expression was analyzed 24 h post infection. NS1 expression in cells infected with plasma that was kept at room temperature was taken as 100%. Non infected cells served as a negative control.

### EXAMPLES

### General Materials and Methods

### Antibodies

The following human monoclonal antibodies were used: 1424 (NS1 specific), 860-55 (VP2 specific) and 1418-16 (VP1 unique region specific), all of which were described by Gigler et al. (25). The VP2-specific antibody hmab8293 was purchased from Millipore.

The labelling of the antibodies with AlexaFluor647® was made with the APEX™ Alexa Fluor 647 Labelling Kit (Invitrogen) according to the manufacturer's instructions.

The generation of Fab fragments from monoclonal antibodies was made with the Pierce FAB Micro Preparation Kit.

### Cells

Whole blood was obtained from healthy B19V seropositive and seronegative volunteers. The study was approved by the Institutional Ethics Committee. Peripheral mononuclear blood cells (PMBCs) were isolated from heparinized whole blood by Ficoll-Paque density gradient centrifugation. Briefly, fresh heparinized blood was mixed with an equal volume of PBS without bivalent ions. Twenty milliliters (ml) of diluted blood was gently layered on 15 ml of Pancoll human in a 50 ml conical tube and centrifuged at 800×g at room temperature for 30 min with no brake. Cells from the lymphocyte layer were collected and washed twice with PBS without bivalent ions. Afterwards the cells were resuspended in the expansion medium.

### Virus and Infection of CD36+ Erythroid Progenitor Cells

A viremic plasma sample containing 1.3×10¹¹ B19V genome equivalents per ml (geq/ml) was derived from a healthy blood donor. The infection was carried out in 24 well plates with 100 μl cell suspension containing 5×10⁵ CD36⁺ cells and 100 μl of a defined B19V concentration per well. The multiplicity of infection (MOI) was considered as genome equivalents per cell.

Regarding the neutralization experiments with the monoclonal antibodies, 100 μl of a defined antibody concentration was added to the 100 μl (5×10⁵) CD36⁺ cells and 100 μl virus solution. To equalize the volume of the wells without antibody solution, 100 μl expansion medium was added.

The cells were incubated for 2 hours on a rocking plate at 4° C. and then expanded with medium to a final volume of 1 ml. After culturing for 24, 48 and 72 hours at 37° in the incubator, cells were harvested and used for cytometric analysis.

### Flow Cytometry

Approximately 1×10⁶ cells were used for flow cytometry analysis at days 0, 5 and 10 of cultivation and the cells were analyzed with the BD FACSCanto™ II flow cytometry system.

For surface staining, the cells were washed once with 2 ml staining buffer (3% FCS 0.1% NaN₃ in PBS; [symbol] 400 g, 5 min) and treated for 20 minutes with fluorescence dye labelled monoclonal antibodies specific for CD34 (PE-Cy7), CD71 (PE), CD36 (APC) and Glycophorin A (PerCP). Globoside P antigen (GloP), the cell surface receptor for parvovirus B19 (59), was detected with by polyclonal rabbit antibodies, followed by anti-rabbit FITC. All stained cells were washed once with 2 ml staining buffer ( [symbol] 400 g, 5 min) and resuspended with 500 μl staining buffer.

For intracellular staining, cells of two wells were combined and thus approximately 1×10⁶ cells were resuspended after a wash step with 500 μl 2% PFA for fixation and were incubated for 15 min in the dark at room temperature. After washing with 2 ml staining buffer ( [symbol] 400 g, 5 min) 10 μl 2% saponin and 3 μl of the AlexaFluor® 647 labelled monoclonal antibodies hmab1424 were added to the runback (approximately 100 μl). After incubation for 30 min in the dark at 4° C., the cells were washed twice with 2 ml 0.1% saponin ( [symbol] 500 g, 5 min) and resuspended in 500 μl 1% PFA.

### Example 1

### Generation of CD36+ Erythroid Progenitor Cells In Vitro

The CD36⁺ erythroid progenitor cells were expanded according to the protocol published by Filippone et al. (48, 24). In brief, the 1×10⁶ PBMCs were cultured for ten days in MEM supplemented with serum substitute BIT9500, diluted 1:5 for a final concentration of 10 mg/ml bovine serum albumin, 10 μg/ml rhu insulin, and 200 μg/ml iron-saturated human transferrin, enriched with 900 ng/ml ferrous sulfate, 90 ng/ml ferric nitrate, 1 μM hydrocortisone, 3 IU/m rhu erythropoietin, 5 ng/ml rhu IL-3 and 100 ng/ml rhu stem cell factor (SCF). The cells were maintained at 37° C. in 5% CO₂.

Upon observation of the initial small clusters on day 5±1, the cells were split to a final concentration of 1×10⁶ cells/ml into their respective media.

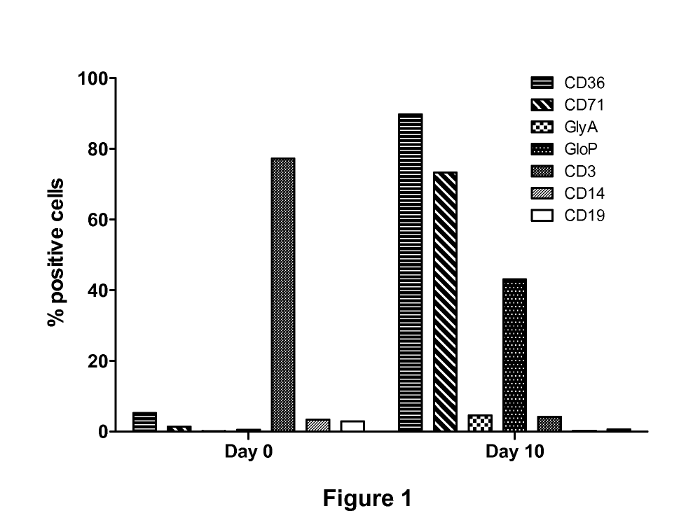

An increase in CD36⁺, CD71⁺, glycophorine A⁺ and globoside P⁺ cells was observed by FACS analysis between day 0 and day 10 of differentiation (FIG. 1). In detail, the initial PBMC population consisted on day 0 of 5.3% CD36⁺, 1.4% CD71⁺, 0.1% glycophorin A⁺, 0.5% globoside P⁺, 77.2% CD3⁺, 3.4% CD14⁺ and 2.9% CD19⁺ cells. On day 10 of differentiation, the cell composition consisted of 89.7% CD36⁺, 73.3% CD71⁺, 4.6% glycophorin A⁺, 43.1% globoside P⁺, 4.2% CD3⁺, 0.2% CD14⁺ and 0.6% CD19⁺ cells.

### Example 2

### Infection of Erythroid Progenitor CD36+ Cells with Parvovirus B19

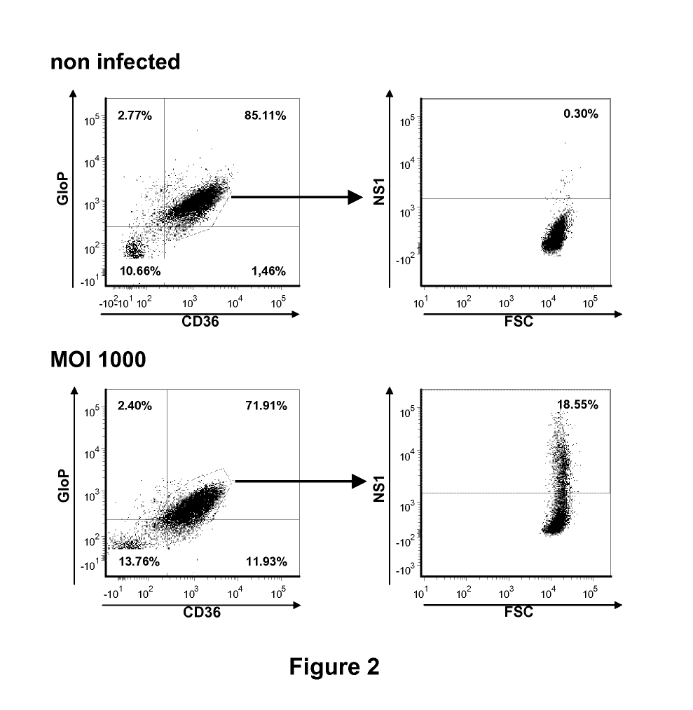

On day 10 of differentiation, 5×10⁵ cells were infected with parvovirus B19 virus and were analyzed for B19V NS1 protein synthesis. Uninfected (FIG. 2, upper panel) and parvovirus B19-infected globoside P+/CD36+ cells (FIG. 2, lower panel) were selected 24 hours post infection (p.i.) (FIG. 2, left-hand side) and NS1-protein synthesis was analysed by FACS using fluorescence-labelled hmab1424 (FIG. 2, right-hand side). Only CD36⁺/Gloside P⁺ cells displayed NS1 protein synthesis. This demonstrates that detection of non-structural proteins is useful for detecting successful infection of cells by B19V (because only the CD36⁺/Gloside P⁺ cells were permissive to infection and only these cells displayed NS1 protein synthesis). Therefore, these methods will be useful for identifying other cells and cell lines that are permissive to B19V infection.

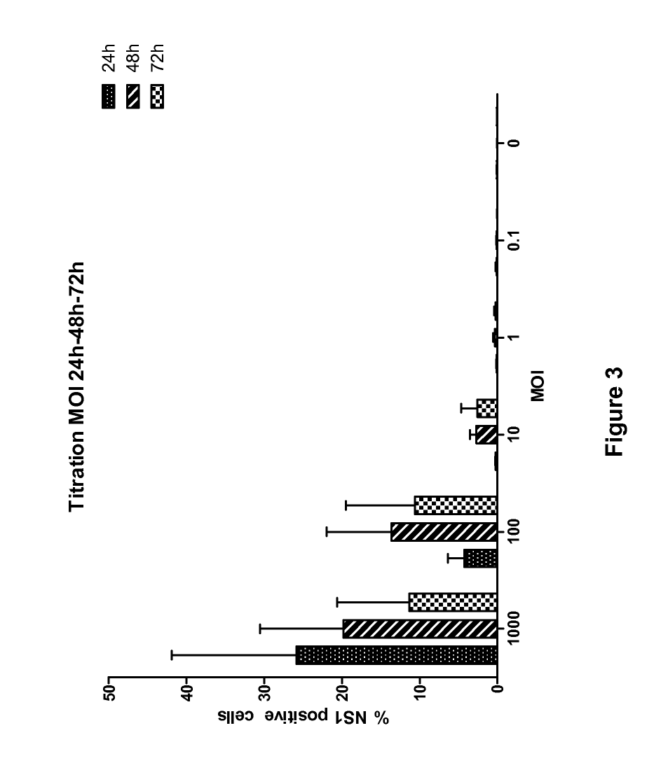

The production of NS1 proteins was also analysed over the course of infection at 24, 48 and 72 hours post infection with a titration of the amount of virus used for infection (FIG. 3). The percentages of NS1-positive cells observed at different MOI/cells and time points p.i. are represented by bars. The MOI was considered to be the number of B19V genome equivalents per cell, thus the cells were infected with a MOI of 1000, 100, 10, 1 and 0.1 (the content of parvovirus B19 genomes in the plasma had been determined beforehand by quantitative PCR). Non-infected cells were used as a control.

24 h post infection at a MOI of 1000, 25.88% of the cells were NS1-positive and the amount declined according to the MOI used for infection: 4.25% (MOI 100), 0.21% (MOI 10), 0.07% (MOI 1) and 0.1% (MOI 0.1).

48 h p.i. at MOI 1000 the percentage of NS1-positive cells was 19.80%, which is a reduction relative to 24 h p.i. At lower MOIs a slight increase to 13.67% (MOI 100), 2.68% (MOI 10), 0.32% (MOI 1) and 0.09% (MOI 0.1) was observed.

72 h p.i. at MOI 1000 the percentage of NS1-positive cells declined and 11.37% (MOI 1000), 10.63% (MOI 100), 2.55% (MOI 10), 0.19% (MOI 1) and 0.02% (MOI 0.1) of cells were detected as NS1-positive. Non infected cells were used as controls and remained negative for NS1 protein synthesis.

The data represent the mean and standard deviation of three independent experiments.

These data demonstrate that detection of non structural proteins enables quantitative analysis of replication-competent parvovirus B19 in a sample.

### Example 3

### Evaluation of the Neutralizing Capacity of B19V Specific Monoclonal Antibodies

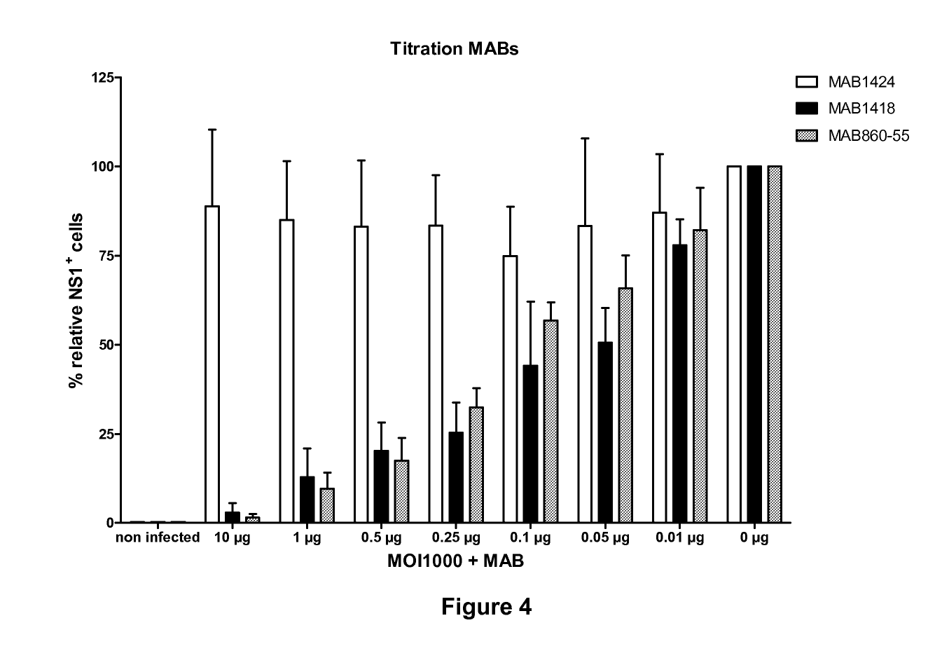

In order to investigate if this read-out system is suitable for the analysis and/or quantification of B19V neutralizing antibodies, in vitro generated CD36+ cells were infected using a MOI 1000/cell with various concentrations of B19V-specific monoclonal antibodies (0.1-10 μg/ml FIG. 4): hmab 860-55 (VP2 specific, grey bars), hmab1418 (VP1 specific, black bars) and hmab1424 (NS1-specific, white bars). Monoclonal antibodies were produced and purified as described previously (25).

24 hours p.i. cells were analyzed for NS1 protein synthesis and the mean percentages of NS1-positive cells were calculated from three independent experiments.

Parvovirus B19 neutralisation was observed using hmab860-55 and hmab1418, whereas hmab1424 showed no inhibition of infection. The amount of NS1-positive cells correlated with the concentration of neutralising antibody employed. Thus, high hmab concentrations resulted in a reduced percentage of NS1-positive cells. For control CD36+ cells were infected with a MOI 1000, but were not incubated with any of the hmab (0 μg/ml). The number of NS1-positive cells detected in this assay was set as 100%. The amount of NS1-positive cells in the cultures treated with monoclonal antibodies was set in relation to this value.

Regarding hmab860-55 (VP2-specific), 1.49%, 9.68%, 17.53%, 32.5%, 56.8%, 65.87% and 82.12% of NS1-positive cells were observed at 24 h p.i. using 10 μg/ml, 1 μg/ml, 0.5 μg/ml, 0.25 μg/ml, 0.1 μg/ml, 0.05 μg/ml and 0.01 μg/ml of purified antibody for virus neutralisation. Regarding hmab1418 (VP1-specific), values of 2.87%, 12.86%, 20.25%, 25.37%, 44.07%, 50.64% and 77.9% NS1-positive cells 24 h p.i. were detected using antibody concentrations of 10 μg/ml, 1 μg/ml, 0.5 μg/ml, 0.25 μg/ml, 0.1 μg/ml, 0.05 μg/ml and 0.01 μg/ml, respectively.

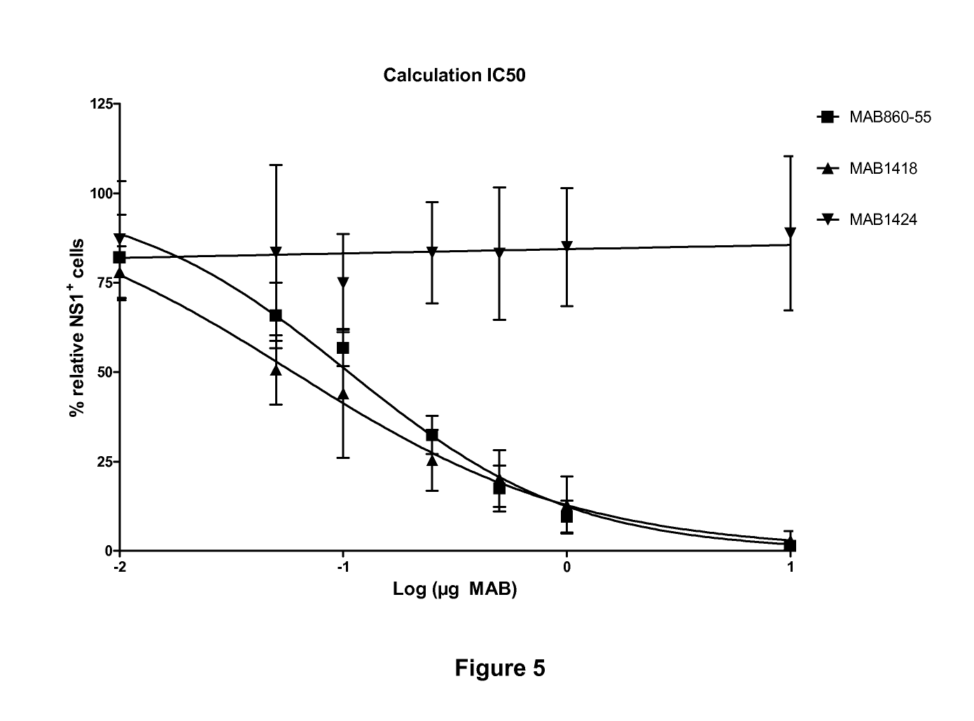

These data demonstrate that the methods of the invention are suitable for analysing and quantifying B19V neutralising antibodies. Here the detection of NS-1 is inversely correlated with the concentration of neutralising antibodies. Using a similar analysis the neutralising capacity of particular antibodies can be analysed and compared, for example through the calculation of IC50 values (see FIG. 5).

These data also demonstrate that antibodies against non-structural proteins, such as NS1-specific hmab1424, do not interfere with infection by parvovirus B19.

### Example 4

### Characterisation of the Parvovirus B19 Neutralizing Capacity of Sera from Seropositive Donors

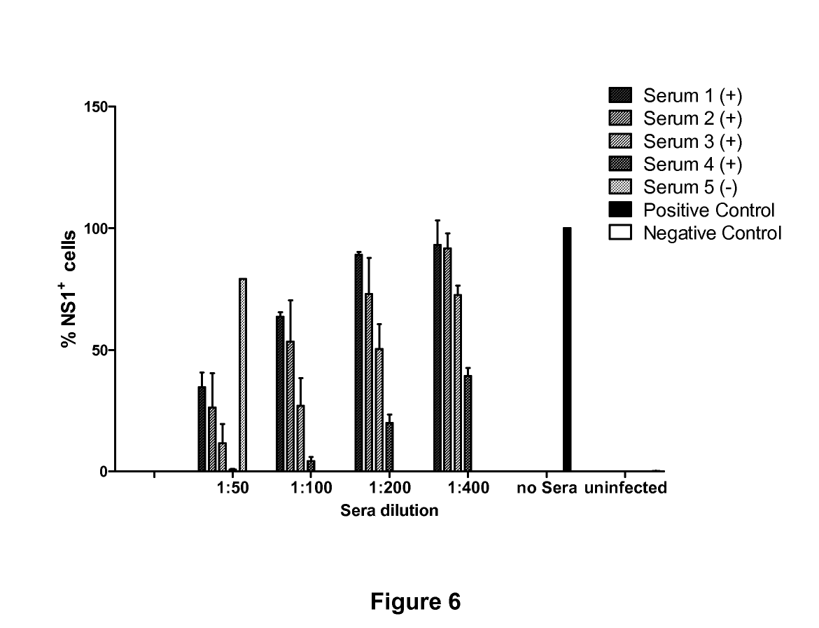

The method of the invention was used to characterise the neutralising capacity of different sera. In vitro generated CD36-positive erythroid progenitor cells were infected with parvovirus B19 using a MOI of 1000/cell. Cells and virus inoculum were co-incubated with various dilutions of sera obtained from four seropositive donors previously infected with B19V (FIG. 6, sera 1-4). As controls cells were incubated with serum obtained from a seronegative donor (serum 5, dilution 1:50, grey bar), were not infected (open bar, not visible) and were incubated without any serum samples (positive control, black bar). The number of NS1-positive cells observed in the positive control was set as 100%. The amount of NS1-positive cells in the cultures incubated with serum samples 1-4 was set in relation to this value.

Parvovirus B19-infected, CD36-positive cells incubated with either the seronegative sample, or without sera displayed NS1-positive cells, thereby indicating the presence of infectious B19V. When sera 1-4 derived from seropositive donors were used, the method of the invention was able to detect neutralizing antibodies, as demonstrated by a reduction in the percentage of NS1-positive cells. Using dilutions of the sera, the neutralizing antibodies present in the four seropositive sera were compared and it was demonstrated that the neutralizing antibody content of serum 4 was greatest.

In detail, serum 1 (blue bar) and 2 (green bar) showed 50% inhibition of infection only at a serum dilution 1:50, whereas serum 3 (orange bar) and 4 (purple bar) showed 50% inhibition at dilutions of 1:100 and 1:400, respectively. Serum 4 (purple bar) displayed the greatest B19V neutralising capacity of greater than 61% inhibition of infection at a dilution of 1:400.

### Example 5

### Effect of Heat Upon the Infectivity of Parvovirus B19

In order to examine a physical inactivation method, we incubated an aliquot of B19V-DNA positive plasma (1×10¹² geq/ml) at different temperatures (room temperature, 40° C., 60° C. and 80° C.) for 5 minutes. Afterwards, in vitro generated CD36⁺ cells were incubated with the pre-treated plasma (MOI 1000).

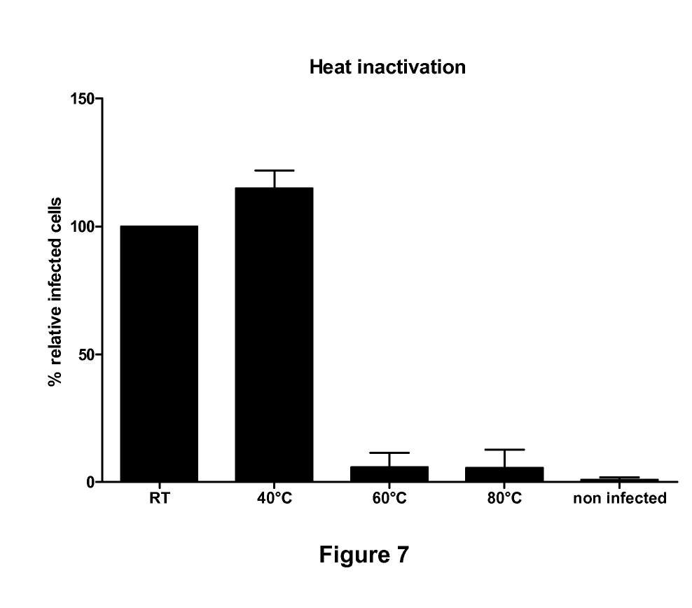

24 h p.i. the cells were analyzed for B19V NS1 protein synthesis (FIG. 7). The number of NS1-positive cells observed in the cultures incubated with the untreated plasma sample (room temperature, RT) in was set as 100%. The amount of NS1-positive cells observed in the cell cultures incubated with heat treated samples was set in relation to this value. Incubating the virus at 40° C. did not alter the infectivity and so 106.45% NS1⁺ cells in relation to the untreated sample were detectable. In contrast, treatment at 60° C. and at 80° C. impaired infection and only 9.00% and 3.8% of the cells, respectively, were NS1-positive. The values represent the mean of three independent experiments.

These data demonstrate that the methods of the invention are able to specifically detect replication-competent parvovirus B19 (here virus that has not been heat inactivated). Results obtained by the methods of the invention are not obscured by the presence of inactive virus particles. Furthermore, these data demonstrate that the methods of the invention are able to assess methods and processes for the inactivation of parvovirus B19.

In addition, these data are significant because they demonstrate that the methods of the invention do not suffer from the deficiencies of conventional methods that rely on the detection of parvovirus B19 DNA. Each aliquot tested comprised 1×10¹² geq/ml of B19V DNA, as determined by qPCR. However, the method of the invention is able to demonstrate that following heat treatment the aliquots heated to 60° C. and at 80° C. comprise very little replication-competent parvovirus B19. Conventional methods that rely on the detection of DNA are not able to detect the differences between the aliquots heated to different temperatures because they all comprise the same amount of DNA. Nor are they suitable for assessing inactivation.

It will be understood that the invention has been described by way of example only and modifications may be made whilst remaining within the scope and spirit of the invention.

### REFERENCES

[1] Modrow (2007) Der Mikrobiologie. 17: 6-15.
[2] Modrow et al. (2010) Molekulare Virologie, 3rd edition (Springer).
[3] Modrow et al. (2010) Medizinische Virologie—Grundlagen Diagnostik, Prävention and Therapie viraler Erkrankungen. 611-623. 2nd edition (Doerr and Gerlich, eds, Thieme).
[4] Young and Brown (2004) N Engl J Med. 350(6): 586-97.
[5] Kerr and Modrow (2006) Human and Primate Parvovirus Infections and Associated Disease. In: Parvoviruses. 385-416 (Kerr et al. eds, Arnold Publishers).
[6] Cotmore et al. (1986) J. Virol. 60(2): 548-57.
[7] Blundell et al. (1987) Virology. 157(2): 534-8.
[8] Doerig et al. (1990) J. Virol. 64(1): 387-96.
[9] St Amand et al. (1991) Virology. 183(1): 133-42.
[10] Luo and Astell (1993) Virology. 195(2): 448-55.
[11] Ozawa (1992) Tanpakushitsu Kakusan Koso. 37(14 Suppl): 2348-54.
[12] Gareus et al. (1998) J. Virol. 72: 509-516
[13] Moffat et al. (1998) J. Virol. 72: 3018-3028
[14] Raab et al. (2002) J. Gen. Virol. 82: 1473-1480
[15] Hsu et al. (2004) Scand J. Infect Dis. 36: 570-577.
[16] Lehmann et al. (2003) Autoimmune Rev. 2(4): 218-23.
[17] Lehman and Modrow (2006) Current Rheumatology Reviews. 2: 159-175.
[18] Modrow and Gärtner (2006) Deutsches Ärtzeblatt. 103(43): A2869-2876.
[19] Tolfvenstam and Brolinden (2009) Semin Fetal Neonatal Med. 14: 218-21.
[20] Liefeldt et al. (2005) J. Med. Virol. 75(1): 161-9.
[21] Modrow et al. (2010) Vox Sanguinis doi: 10.1111/j.1423-0410.2010.01445.x
[22] Blümel et al. (2005) J. Virol. 79(22): 14197-206.
[23] Hemauer et al. (1999) J. Gen. Virol. 80 (Pt 3) :627-30.
[24] Wong et al. (2008) J. Virol. 82(5): 2470-6.
[25] Gigler et al. (1999) J. Virol. 73(3): 1974-9.
[26] Ros et al. (2006) Virology. 345: 137-147.
[27] Boschetti et al. (2004) Transfusion. 44(7): 1079-86.
[28] Yunoki et al. (2003) Vox Sang. 84(3): 164-9.
[29] Rosenfeld et al. (1992) J Clin Invest. 89(6): 2023-9.
[30] Qiu et al. (2007) J. Virology. 81(21): 12080-12085.
[31] Takahashi et al. (1989) J. Infect. Dis. 160(3): 548-9.
[32] Takahashi et al. (1993) Arch. Virol. 131(1-2): 201-8.
[33] Miyagawa et al. (1999) J. Virol. Methods. 83(1-2): 45-54.
[34] Munshi et al. (1993) J. Virol. 67(1): 562-6.
[35] Shimomura et al. (1992) Blood. 79(1): 18-24.
[36] Morita et al. (2001) J. Virol. 75(16): 7555-63.
[37] Wong and Brown (2006) J. Clin. Virol. 35(4): 407-13.
[38] Ozawa et al. (1986) Science. 233(4766): 883-6.
[39] Ozawa et al. (1987) Blood. 70(2): 384-91.
[40] Srivastava and Lu (1988) J. Virol. 62(8): 3059-63.
[41] Schwarz et al. (1992) J. Virol. 66(2): 1273-6.
[42] Srivastava et al. (1992) Virology. 189(2): 456-61.
[43] Brown et al. (1991) J. Gen. Virol. 72(Pt 3): 741-5.
[44] Yaegashi et al. (1989) J. Virol. 63(6): 2422-6.
[45] Wong et al. (2007) J. Virol. 82(5): 2470-6.
[46] Freyssinier et al. (1999) Br. J. Haematol. 106(4): 912-22.
[47] Giarratana et al. (2005) Nat. Biotechnol. 23(1): 69-74
[48] Filippone et al. (2010) PLoS One. 5(3): e9496.
[49] WO 2009/109604
[50] Methods In Enzymology (S. Colowick and N. Kaplan, eds., Academic Press, Inc.)
[51] Handbook of Experimental Immunology, Vols. I-IV (D. M. Weir and C. C. Blackwell, eds, 1986, Blackwell Scientific Publications)
[52] Sambrook et al. (2001) Molecular Cloning: A Laboratory Manual, 3rd edition (Cold Spring Harbor Laboratory Press).
[53] Handbook of Surface and Colloidal Chemistry (Birdi, K. S. ed., CRC Press, 1997)
[54] Ausubel et al. (eds) (2002) Short protocols in molecular biology, 5th edition (Current Protocols).
[55] Molecular Biology Techniques: An Intensive Laboratory Course, (Ream et al., eds., 1998, Academic Press)
[56] PCR (Introduction to Biotechniques Series), 2nd ed. (Newton & Graham eds., 1997, Springer Verlag)
[57] Current Protocols in Molecular Biology (F. M. Ausubel et al., eds., 1987) Supplement 30
[58] Smith & Waterman (1981) Adv. Appl. Math. 2: 482-489.
[59] Brown et al. (1993) Science. 262(5130): 114-7.

1. (canceled)

2. A method for the detection of parvovirus B 19 in a sample, comprising steps of: (i) contacting the sample with cells which can be infected by parvovirus B19; (ii) incubating the cells; and (iii) determining the presence of parvovirus B19 non-structural protein.

3. (canceled)

4. The method according to claim 2, wherein the sample is a blood sample, a blood plasma product or a parvovirus B19 vaccine composition.

5. A method for testing a pharmaceutical product, comprising steps of (i) contacting the product or a sample of the product with cells which can be infected by parvovirus B19; (ii) incubating the cells; and (iii) determining the presence or absence of parvovirus B19 non-structural protein.

6. The method of claim 5, wherein in the pharmaceutical product is a parvovirus B19 vaccine composition or a blood plasma product such as a coagulation factor product, a serum albumin product, or an immunoglobulin preparation.

7. A method for manufacturing a blood product, comprising steps of (i) contacting a blood sample or part of a blood sample with cells which can be infected by parvovirus B19; (ii) incubating the cells; (iii) determining the presence or absence of parvovirus B 19 non-structural protein; and (iv) accepting a blood sample for inclusion in the blood product if parvovirus B19 non-structural protein is determined to be absent.

8. The method of claim 7, wherein the blood product is a coagulation factor product, a serum albumin product, or an immunoglobulin preparation.

9. The method of claim 2, wherein the non-structural protein is NS 1.

10. The method of claim 2, wherein the non-structural protein is detected with antibody.

11. The method of claim 10, wherein the antibody is labelled.

12. (canceled)

13. (canceled)

14. (canceled)

15. (canceled)

16. The method of claim 5, wherein the non-structural protein is NS 1.

17. The method of claim 7, wherein the non-structural protein is NS 1.

18. The method of claim 5, wherein the non-structural protein is detected with an antibody.

19. The method of claim 7, wherein the non-structural protein is detected with an antibody.

20. The method of claim 18, wherein the antibody is labeled.

21. The method of claim 19, wherein the antibody is labeled.
